(19) United States
(12) Patent Application Publication
(10) Pub. No.: US 2023/0072482 A1
(43) Pub. Date: Mar. 9, 2023

(54) SPECULUM AND OTOSCOPE

(71) Applicant: Audelation Limited, London (GB)

(72) Inventor: Tsong KWONG, London (GB)

(21) Appl. No.: 17/798,515

(22) PCT Filed: Feb. 10, 2021
(86) PCT No.: PCT/GB2021/050305

§ 371 Date: Aug. 9, 2022

(30) Foreign Application Priority Data
Feb. 10, 2020 (GB) 2001741.4

(51) Int. Cl. A61B 1/227; A61B 1/05; A61B 1/12; A61B 1/00; A61B 1/06; A61B 1/32

(52) U.S. Cl. CPC A61B 1/227; A61B 1/05; A61B 1/128; A61B 1/0017; A61B 1/0669; A61B 1/0684; A61B 1/32

(57) ABSTRACT

A speculum for use in an otoscope, wherein the speculum (1D) comprises a body having a passageway therethrough, defining a first end with a first opening, and a second end (1F) with a second opening; the speculum further comprising a light source (1B); the body of the speculum (1D) comprising a light transmitting portion; in which the light source (1B) is coupled to the light transmitting portion at least one coupling point (1C) such that the light transmitting portion is arranged to transmit light through the body from each coupling point (1C) to the second end (1F) of the speculum.

### RELATED APPLICATION INFORMATION

This patent claims priority from International PCT Patent Application No. PCT/GB2021/050305, filed Feb. 10, 2021, entitled, “SPECULUM AND OTOSCOPE”, which claims priority to United Kingdom Patent Application No. 2001741.4, filed Feb. 10, 2020, all of which are incorporated herein by reference in their entirety.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### FIELD OF THE INVENTION

The present invention relates to a speculum and an otoscope.

### BACKGROUND OF THE INVENTION

The ears of humans and other animals can require examination and/or treatment from time to time, or when the patient is afflicted with a particular disease. It is especially common for patients to require removal of earwax, which is also known as cerumen, using techniques such as aural microsuction.

As well as being useful in the restoration of a patient's hearing and for general aural health, the removal of earwax is necessary in audiological assessments, and in the fitting and maintenance of hearing aids. The removal of wax or foreign bodies from the ear of a patient is the most common outpatient appointment in the world.

The overall process of removing wax or a foreign body from a patient's ear is typically composed of two procedures: ear canal inspection (otoscopy); and removal of the wax or foreign body using microsuction and/or tools, such as wax hooks and/or Jobson Horne Probes. The classical technique is for the clinician to hold a speculum, which is typically a lightweight plastic cone, in their non-dominant hand and uses the instruments listed above and inserts the tools through the centre of the speculum to remove the contents of outer ear canals such as wax.

Examination and treatment of the outer ear canal can be performed by a variety of devices, such as microscopes, loupe glasses, rigid endoscopes and conventional otoscopes. Tympa Health Technologies Limited of London, United Kingdom, have proposed a smartphone-based system using a smartphone with a camera; a similar smartphone-based system is described in WO2019/116024. We ourselves have proposed camera-based solutions in our earlier PCT application published as WO2018/197870; we refer to that application below as “our earlier application” and the device described therein as “our earlier device”. However, each of these types of device can be improved.

Ear microscopes are typically preferred for the removal of wax or a foreign body from a patient's ear. These devices allow for direct vision of the ear canal. Ear microscopes are typically freestanding. However, ear microscopes are large and not portable, occupying a lot of clinic space. Ear microscopes are also considerably expensive. In addition, ear microscopes do not allow the patient to move position during examination and/or treatment, and a microscope can require frequent readjustment during an examination or treatment to keep the desired area in view.

Loupe glasses are glasses with small, magnifying lenses that are very portable and allow visualisation of the ear canal. Loupe glasses suffer from the fact that they need to be bespoke for each clinician, to fit their eye dimensions and prescription. In addition, loupe glasses provide a fixed focus, magnification and viewpoint, meaning that the clinician has to move their head to produce different views. This requirement for movement can make clinicians nauseous. Loupe glasses commonly require illumination and are therefore used with headwear and a large battery pack to provide a light source. A clinician using loupe glasses must manoeuvre around the patient to keep the area of examination and/or treatment in view.

Rigid endoscopes and conventional otoscopes offer visualisation of the ear canal but restrict or even prevent the access of instruments required to perform treatments such as aural microsuction.

In particular, rigid endoscopes require a lighting source to be present within the ear canal. Such lighting sources typically generate heat, which puts the patient at risk of receiving burns. Furthermore, in use the endoscope extends into the ear canal, restricting the movement of instruments and providing an increased risk of ear drum perforation. The insertion of rigid endoscopes into the ear canal also means that they require sterilisation after each use to prevent cross-contamination between patients.

Conventional otoscopes are frequently used to physically examine the outer ear canal. Otoscopes typically comprise a handle and viewing system, wherein the viewing system holds a light source and a tip. Otoscopes are usually used with disposable ear specula to cover the tip and prevent cross-contamination between patients. However, having a speculum attached to, for example screwed on to, and covering the tip prevents the passage of instruments into the ear canal to perform treatments, such as earwax removal.

As referred to above, our earlier device described in WO2018/197870 describes a digital camera system that allows direct visualization of the outer ear canal and tympanic membrane/eardrum through an ear speculum and allows simultaneous passage of instruments through the outer and inner ring of the speculum to perform procedures such as microsuction and ear procedures. Our earlier device is attached either by wire or wireless connections to a display such as a computer tablet or computer. Lighting can be provided at the end of the speculum furthest from the patient, shining into the ear.

### SUMMARY OF THE INVENTION

The inventor has realised that the lack of portability of ear microscopes prevents these devices being used outside of the clinic. This presents a particular issue because patients must be referred to a specialist that has the required equipment. Furthermore, the lack of portability of the equipment means that treatment cannot easily be performed on patients that are immobile.

In addition improved visualisation of the outer ear canal and ear drum whilst also allowing access for instruments that are required to be inserted in to the outer ear canal is an aim of this invention.

Therefore, the inventor has determined a need for an otoscope that is portable to enable clinicians to perform procedures such as earwax removal outside of the clinic and that allows visualisation of the outer ear canal and ear drum without preventing, and ideally without unduly restricting, access of instruments.

The present invention provides, in a first aspect, a speculum for use in an otoscope, wherein:
the speculum comprises a body having a passageway therethrough, defining a first end with a first opening, and a second end with a second opening; the speculum further comprising a light source;
the body of the speculum comprising a light transmitting portion;
in which the light source is coupled to the light transmitting portion at at least one coupling point such that the light transmitting portion is arranged to transmit light through the body from each coupling point to the second end of the speculum.

Preferably, each coupling point is at the first end of the speculum; this allows the light source to be as far from the patient (who will be at the second end in use) as possible.

One of the issues with our earlier device is related to the use of a lighting structure where there are a ring of LEDs at the proximal end of the speculum from the patient, there being insufficient room to provide the LEDs to the distal end. Having a ring of LEDs at a distance from the patient can mean however that light is lost from the point from illumination on the light source to the tip of the speculum and beyond.

By illuminating from the tip (having transmitted the light from elsewhere) means
Better illumination because the light source is closer to the ear canal, tissues, blood vessels and tympanic membrane
Less powerful lighting can be used
Possibility to slim down and make more compact the device by removing the LED ring system
Reduce the need for any focusing lenses in front the lighting structure as we describe in our earlier application

### Light Pipe Concept

The light transmitting portion may comprise a transparent material typically covered in a cladding material. As such, the light transmitting portion may act as a light pipe. The cladding material may act to guide the light within the transparent material and may have a reflective inner surface in contact with the transparent material. Alternatively, the cladding material and the transparent material may have refractive indices such that total internal reflection of the light from the light source occurs at a junction between the transparent material and the cladding material (as occurs in a fibre optic cable). The cladding material may block transmission of light through itself.

The light transmitting portion may have a first cross-sectional area at at least one coupling point, and a second cross-sectional area at the second end, with the second cross-sectional area being smaller than the first cross-sectional area. As such, this may act to concentrate the light passed through the light transmitting portion.

The speculum may comprise at least one light sensor arranged to determine where light is being emitted from the second end, and a control circuit coupled to each light sensor, the control circuit being arranged to modulate the brightness of the light emitted by the light source in response to light levels detected by each light sensor.

The light sensor may comprise a digital camera arranged to capture images through the passageway through the body of the speculum. As such, the control circuit may be arranged so as to analyse images captured by the digital camera and to determine illumination levels of at least one, if not a plurality, of regions of those images.

Where the light source comprises a plurality of light generating elements, the control circuit may be arranged so as to dim or extinguish any light generating element to a region where each light sensor indicates no light is being emitted (so as to reduce or cease transmitting to an area of the second end which is blocked in some way), and optionally to increase the brightness of light generating element transmitting to a region which each light sensor indicates light is being transmitted (so as to accommodate for the blockage). Typically, the control circuit will disregard any short term (e.g. less than a predetermined period, such as 1 s, 0.5 s, 0.1 s) change in the light levels indicated by each light sensor, so that temporary blockages (such as hairs moving past the end of the speculum) do not affect the illumination.

The speculum may be provided with a housing. The housing may house at least one of the light source, the control circuit and at least part of a digital camera. If the digital camera is provided in the housing, the speculum can by itself function as an otoscope.

The digital camera may comprise an image sensor and image processing circuitry. The housing may be formed of first and second parts pivotably mounted with respect to each other. The first part may house the image sensor, and so may be provided with a viewing window to view the passageway through the body of the speculum. The second part may house the image processing circuitry. There may be a flexible connection connecting the image sensor and the image processing circuitry, which may comprise at least one wire.

According to a second aspect of the invention, we provide an otoscope, comprising a digital camera, a speculum in accordance with the first aspect of the invention and an attachment means for attaching the camera to the speculum, and wherein the attachment means is for attaching to the first end of the speculum.

According to a third aspect of the invention, we provide a speculum for use in an otoscope, wherein the speculum comprises a body having a passageway therethrough, defining a first end with a first opening, and a second end with a second opening; and a housing at least part of a digital camera;
the digital camera comprising an image sensor and image processing circuitry, the housing being formed of first and second parts pivotably mounted with respect to each other, with the first part housing the image sensor and the second part housing the image processing circuitry.

The first part may be provided with a viewing window to view the passageway through the body of the speculum. There may be a flexible connection connecting the image sensor and the image processing circuitry, which may comprise at least one wire.

### DETAILED DESCRIPTION OF THE INVENTION

There follows a description of embodiments of the invention, described with reference to the accompanying drawings, in which:

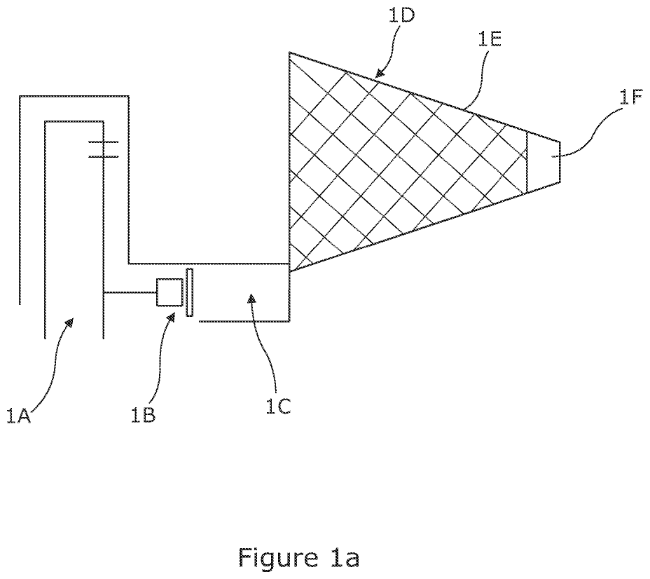

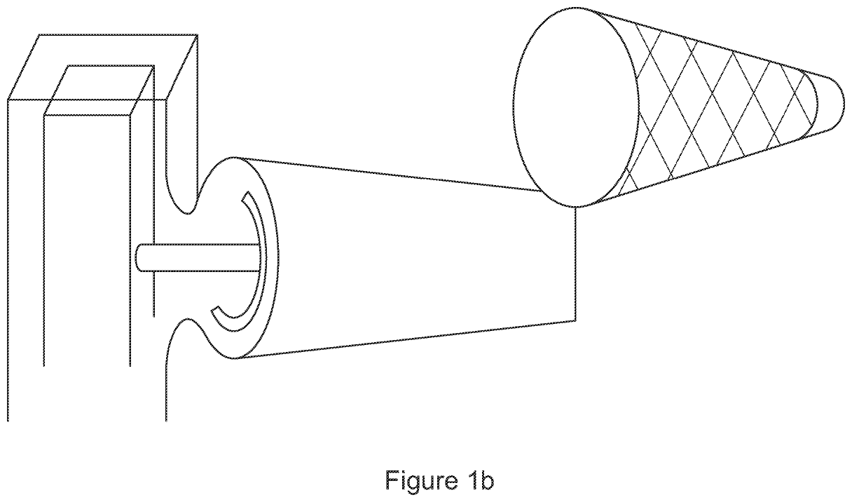

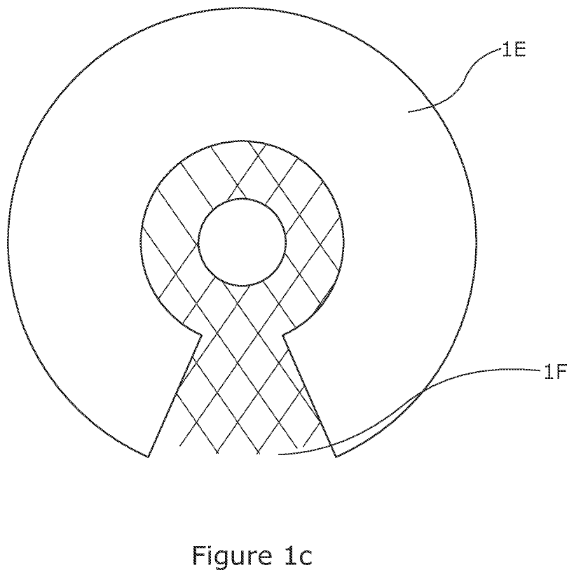

FIGS. 1a to 1c show an otoscope in accordance with a first embodiment of the invention, in side elevation, perspective view and front elevation respectively.

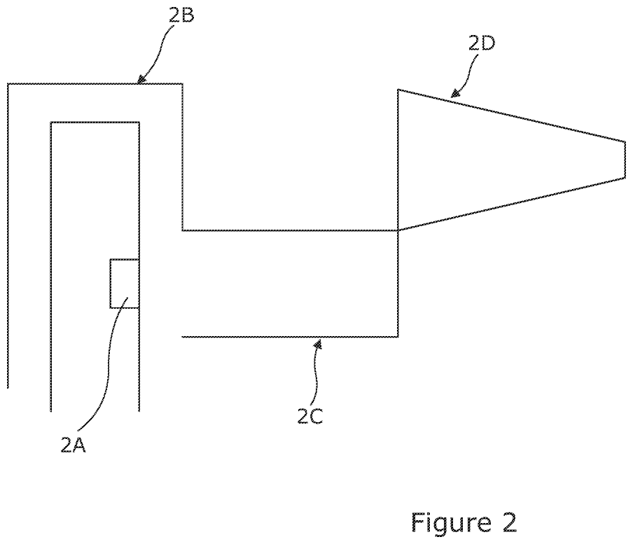

FIG. 2 shows a schematic side view of an otoscope in accordance with a second embodiment of the invention; and

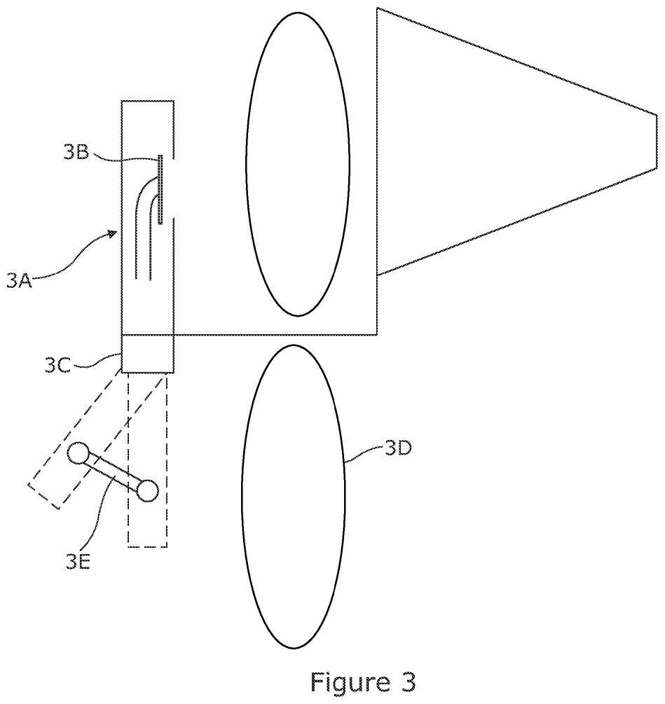

FIG. 3 shows a schematic side view of an otoscope in accordance with a third embodiment of the invention.

### DESCRIPTION OF APPARATUS

In a first embodiment of the invention, an otoscope with a speculum 1D is shown in FIGS. 1a to 1c of the accompanying drawings. In this embodiment, 1A refers to a housing of the form of a tower, hereafter the camera tower and a protrusion (1B) that sticks out from the tower. The protrusion forms a curved shape that may be circular but not restricted to that shape which contains an LED or row of LEDs that provide illumination. The speculum has been redesigned to allow a cover over the lights.

The LEDs are positioned so the light is directed to a bridge 1C joining the protrusion to the speculum 1D. The speculum bridge 1C is made of a material that can conduct light (e.g. a transparent plastic). The conducted light then is directed to the cone of the speculum 1D. The speculum is again constructed of a material that conducts light (e.g. a transparent plastic). It is envisioned that light concentrates at the tip of the speculum—to create what we call the “light emitting tip” (LET) 1F. The speculum defines an internal bore through which a user can observe, and narrows both internally and externally from the end at which it is connected to the bridge to the light emitting tip 1F.

The LEDS can be powered by electronics (and a power source such as a battery) from within the tower and can be controlled for dimming/brightening.

So that light is not emitted during the transfer to the tip, the speculum 1D can be manufactured so that it is spray painted black or another dark colour on its surface 1E. We make specific reference to the tip 1F, which is devoid of any black paint or material. By being devoid of material, the light is then “focused” to the tip of the speculum and so projects into the ear canal (when the ear speculum is inserted). The paint free tip 1F can be manufactured by having a cover that is inserted into distal end of the speculum that may cover the tip and a certain length from the tip to keep that part of the speculum tip pain free.

The cone part of the speculum can be constructed also from a combination of nonconductive and conductive material that is either mixed or proportion of the circumference of the material. By doing it this way, can reduce the cost of the speculum.

When LEDs are powerful enough, only part of the distal circle of the speculum may be illuminated that can be powerful enough provide full illumination of the ear

It is envisioned the colour the LED is primarily white and cold in temperature however a combination of different coloured LED to combine to form a white light or other desired light is also described including as ultra-violent bandwidth or infrared or anywhere else on the electromagnetic spectrum. In particular having infrared sensors in a body cavity with no natural illumination may improve image quality.

The ear canal is not a straight structure and has many undulations and curves, ear hair, wax and debris. Because of the tip of the speculum has the light emitting element or tip “LET”, it can be envisioned that part of the light emitting element can be partially blocked. We describe a system where the light emitting diodes also have a sensor or plurality of illumination detecting sensors built in that detect if a part of the light emitting element is blocked. A simple algorithm can be that system can calculate how much light is being emitted, make deductions for any light lost during the flow of light to the light emitting tip and compare how much light comes back and making corrections to provide better illumination. We also describe the system that can compensate for when light emitting tip is blocked by or a combination of:
1. Switching off the lighting source which supplies the affected area.
2. Boosting the other lighting sources to compensate.
3. A dynamic intelligent system where very temporary blockages e.g. moving past an ear hair do not cause changes to the illumination, but longer blockages trigger change in the illumination.

There may be a light control for the light source arranged to vary a brightness of the light output from the light source. The light control may vary the brightness over at least three discrete levels, or continuously over a range of light levels. The light control may be manually activated and/or may be automatic based upon ambient light levels and the output of the light sensor.

While we describe a system with primarily light sensors and illumination, there is no restriction in the use of other sensors, energy emitting devices (e.g. speakers). One such example is the combination with supplementary camera sensors which with the main camera can form 3D images. A further example is the use of the speaker (or other sound emitting device) to emit sounds for use in audiometry testing.

In a second embodiment of the invention shown in FIG. 2 of the accompanying drawings, the lighting source 2A is in the tower 2B and not in the protrusion outline 1B in FIG. 1. This configuration has an advantage that the lighting source 2A is entirely contained in the tower 2B and so easier to protect from infection risk aspect.

The lighting from 2A is alignment with the bridge 2C connecting to the speculum 2D. The lighting from 2A can be a single LED or a multitude of LEDs. The lighting system is in direct line of the bridge which conducts light (through a transparent medium) to the speculum. There may be a layer between the lighting system and the bridge that helps to focus or intensify the light down the bridge.

The LEDs can be powered by electronics from the tower and can be controlled for dimming/brightening purposes.

A third embodiment of the invention is shown in FIG. 3 of the accompanying drawings. In the prior art, a classical camera sensor would normally be attached and wired to an electronics board. In this embodiment, we use an electronics boards where the camera sensor 3B is off the main board and be positioned as desired.

This embodiment of the camera can avoid the use of reflective mirrors. The aperture of the camera can be smaller as a result which results in a smaller tower 3A.

One of the advantages of a flex-camera system is that it is a mirror-less/minimum mirrors but not necessarily lens-less system. Having a mirror system means there must be alignment of the mirrors. In our previous device, one embodiment has a mirror system to get image to the camera. However, having housing for the mirror system creates some physical restrictions on the device. One way of holding our previous device is to hold it like a pencil, where in the non-dominant hand the edge of the speculum is held with fingers and thumb for control. However the housing for the mirror system can get in the way for some hands—making it uncomfortable to use.

Having a mirror-less/minimum mirror system allows for a housing that can adapt the size of the user's hands. FIG. 3 shows a hinge like mechanism 3C along the housing to accommodate the size of the user's hands. In this embodiment the hinge can be at the base of the tower and the electronics to allow a swinging mechanism out to accommodate the size of the hand 3D. The hinge 3C can be strengthened since it is envisaged it will need to tolerate thousands of swings. The hand size can determine how far the swing mechanism 3E must rotate to accommodate the hand.

We also describe a dynamic/intelligent illumination/video system as similarly described in FIGS. 1a to 1c.

In this dynamic learning system features include:
Variable illumination including the reduction and boosting of the lighting LEDs according to the light feedback to the camera sensor. This can be particularly useful for views near the speculum which are at risk of being “over-illuminated” and poor image interpretation by the software. Typically to prevent overflooding of the camera sensor, digital cameras have a AGC (Automatic Gain Control) however this can take time and may not function.
Though the system is designed to vary light levels automatically, we also propose a mechanism such as a switch to manually vary the light intensity.
Integration of infrared sensors that integrate with the light sensors to optimise the clarity of imaging.
A stereoscopic element can sit over the camera sensor and lighting as an alternative to create 3D vision.
Live/near-simultaneous image modification/manipulation by the interpreting software to optimise viewing.
The ability to zoom in/zoom out of a live image and the physical controls that allows to do that on our previous device or a display unit.
In addition, to the software manipulation—an additional feature can be for automatic recognition of the images which may be relayed back to software which can also control/manage the patient record, remove image viewing by professionals.

Other features of the devices of the preceding embodiments include:
Integration with the anti-infective measures as listed in our earlier application to prevent contamination of the LEDS during the microsuction procedure
Multicoloured LEDS where the light can be blended to form a colour of light that optimal to viewing human tissues
A control mechanism to control the brightness of the light can be controlled by the non-dominant hand or the tablet that may dim or brighten the light, turn some LEDS off or a mixture of both.
A filter mechanism in front the LEDS to filter unwanted wave lengths of light that are impede good image quality
A silencer device—if using in particular an autofocus camera, the camera will be automatically adjusting itself. In the closed space of the ear, that can create unwanted noise that maybe irritating to the patient.
A sound emitting device that can be used as a form of pure tone audiometry and may even use the sound of the camera as part of that testing
A ribbed edge to the handheld portion of the speculum that allows for better handling of the speculum.
An orientation sensor such as a gyroscope and associated hardware and associated software to correct for issues with orientation when rotating the camera that is ideally part of the internal electronics. It is important for practitioners that they are fully aware of the spatial geometry of the tympanic membrane. One of the original issues of our previous device is that if you turn the camera, the orientation of image seen on the camera will change as well. As such, the device may be arranged so as to keep the image output consistently oriented as the device is rotated, or may flip the image through 180 degrees (or some other increment, such as 90 degrees) once the device has been rotated through 180 degrees (or the increment) from a datum position. A marker on the screen can also aid in the orientation of the image.
A fan and cooling system that allows the electronics to stay cool during its use. This may be connected the outer housing the device to allow the outer housing to cool and feel cool during use. The outer housing is also of such a shape to be able to be received by a cover which would help to insulate the users hand away from heat.
Variable Focus Feature where there are moving electro-mechanical/mechanical system of lens or set of lenses with a control mechanism or liquid lens system where variable focus from the tip of the speculum is possible at any length from speculum distal end. This could be automated with integration into the intelligent lighting system or independently.
In FIG. 10 of our earlier application, we describe a smartphone embodiment of our previous device which has features such as a gap between the smartphone and the speculum attachment to allow instruments to pass in this space and to go through the speculum to allow procedures such as microsuction to be done.
   One of the issues of this embodiment it can be very bulky and may need a handle to securely use the device. However, the typical procedure of microsuction needs manipulation by pulling of the ear lobe to straighten the canal.
   We propose a clip like device that attaches to the devices and simultaneous attachment to the ear lobe such as soft clamp and gentle tension can be created by device to pull the ear canal to straighten it. Tension can be created but limited to by ratchet mechanism or graded length mechanism (similar to changing the length of walking sticks)

We propose various embodiments of the improved device that may have all or part of the features proposed above.

There now follows the text of our previous application, which describes further aspects and embodiments of the invention.

In April 2017, the company filed an application for a new way of visualising the outer ear canal and performing outer ear procedures. This shall be called and referenced as Device GWMV. Device-GWMV describes digital camera system that allows direct visualization of the outer ear canal and tympanic membrane/eardrum through an ear speculum and allows the passage of instruments through the outer and inner ring of the speculum to perform procedures such as microsuction and ear procedures. The Device-GWMV is attached either by wire or wireless connections to a display such as a computer tablet or computer.

The inventor has realised that the portability of ear microscopes prevents these devices being used outside of the clinic. This presents a particular issue because patients must be referred to a specialist that has the required equipment. Furthermore, the lack of portability of the equipment means that treatment cannot easily be performed on patients that are immobile.

In addition improved visualisation of the outer ear canal and ear drum whilst also allowing access for instruments that are required to be inserted in to the outer ear canal is an aim of this invention.

Therefore, the inventor has determined a need for an otoscope that is portable to enable clinicians to perform procedures such as earwax removal outside of the clinic and that allows visualisation of the outer ear canal and ear drum without preventing, and ideally without unduly restricting, access of instruments.

One of the issues of Device-GWMV is that the current embodiments as described has a lighting structure where there is a ring of LED's around an aperture on the tower. Having a ring of LEDs can mean however that light is lost from the point from illumination on the light source to the tip of the speculum and beyond.

We propose a new way of improving the illumination and the optics of the Device-GWMV by using light-pipe technology to transmit light to the tip of the ear speculum. By having the illumination at the tip of the speculum means
Better illumination because of the light source is closer to the ear canal, tissues, blood vessels and tympanic membrane
Less powerful lighting can be used
Slim down and make more compact the tower by removing the LED ring system

The present invention provides, in a first aspect, an otoscope comprising a digital camera, a speculum and an attachment means for attaching the camera to the speculum, wherein:
the speculum defines a first end with a first opening, and a second end with a second opening; and wherein the attachment means is for attaching to the first end of the speculum;
the camera is has a first position substantially in the plane of the first opening such that it can view a region beyond the second opening.

The inventor has devised a portable otoscope that is capable of use in a multitude of different scenarios. The otoscope allows visualisation of the outer ear canal and ear drum without preventing, and ideally without unduly restricting, access of instruments. The otoscope of the present invention is simply and low cost.

The present invention is directed to improving the construction of otoscopes and its primary function is to allow digital viewing of the outer canal and ear drum. The inventor proposes the use of a handle-less otoscope. We describe in the present invention integration with a digital camera and ear speculum and a novel mechanism to avoid wax and debris contamination. Typically otoscopes require the construction of a handle to be able to hold a speculum—however in this invention the inventor describes how an otoscopy can be performed by holding the speculum alone.

In one embodiment the camera is configured to be displaced from the detection position. Such displacement may aid the access and/or removal of the instrument. In particular, such displacement may help to prevent contaminated instruments from contaminating parts of the otoscope that are not disposable, such as the camera.

The otoscope of the present invention may suitably be used in the process of removing earwax or debris from the outer ear canal.

Accordingly, in a second aspect, the present invention provides a method of using an otoscope according to the first aspect.

According to a third aspect, the present invention provides a method of diagnosing the human or animal with a disease or condition, the method comprising providing an otoscope according to the first aspect and using the otoscope in the process of diagnosing the human or animal body with a disease or condition.

According to a fourth aspect, the present invention provides a method of treating the human or animal body, the method comprising providing an otoscope according to the first aspect and using the otoscope in the process of treating the human or animal body.

### DETAILED DESCRIPTION OF THE INVENTION

### FIG. 1: Embodiment of a Speculum, Camera Unit and Speculum and Lighting at the End

In this embodiment; 1A refers to the camera tower and protrusion (1B) that sticks out. The protrusion forms a curved shape that may be circular but not restricted to that shape which contains an LED or row of LEDs that provide illumination. The speculum has been redesigned to allow a cover over the lights.

The LEDs are positioned so the light is directed to the bridge 1C of the speculum. The speculum bridge is made of a material that can conduct light. The conducted light then is directed to the cone of the speculum 1D. The speculum is again constructed of a material that conducts light. It is envisioned that light concentrates at the tip of the speculum—to create what we call the “light emitting tip” (LET)

The LEDS can be powered by electronics from the tower and can be controlled by a for dimming/brightening.

So that light is not emitted during the transfer to the tip, the speculum can be manufactured so that it is spray painted black or another dark colour (1E). We make specific reference to the tip 1F, which is devoid of any black paint or material. By being devoid of material, the light is then “focused” to the tip of the speculum and so projects into the ear canal (when the ear speculum is inserted). The paint free tip can be manufactured by having a cover that is inserted into distal end of the speculum that may cover the tip and a certain length from the tip to keep that part of the speculum tip pain free.

The cone part of the speculum can be constructed also from a combination of nonconductive and conductive material that is either mixed or proportion of the circumference of the material. By doing it this way, can reduce the cost of the speculum.

When LEDs are powerful enough, only part of the distal circle of the speculum may be illuminated that can be powerful enough provide full illumination of the ear

It is envisioned the colour the LED is primarily white and cold in temperature however a combination of different coloured LED to combine to form a white light or other desired light is also described including as ultra-violent bandwidth or infrared or anywhere else on the electromagnetic spectrum. In particular having infrared sensors in a body cavity with no natural illumination may improve image quality.

The ear canal is not a straight structure and has many undulations and curves, ear hair, wax and debris. Because of the tip of the speculum has the light emitting element “LET”, it can be envisioned that part of the light emitting element can be partially blocked. We describe a system where the light emitting diodes also have a sensor or plurality of illumination detecting sensors built in that detect if a part of the light emitting element is blocked. A simple algorithm can be that system can calculate how much light is being emitted, make deductions for any light lost during the flow of light to the light emitting tip and compare how much light comes back and making corrections to provide better illumination. We also describe the system that can compensate for when light emitting tip is blocked by or a combination of:
1. Switching off the lighting source which supplies the affected area
2. Boosting the other lighting sources to compensate
3. A dynamic intelligent system where very temporary blockages e.g. moving past an ear hair do not cause changes to the illumination but longer blockages trigger change in the illumination

While we describe a system with primarily light sensors and illumination, there is no restriction in the use of other sensors, energy emitting devices (e.g. speakers). One such example is the combination with supplementary camera sensors which with the main camera can form 3D images.

### FIG. 2: A Second Embodiment of the Device-GWMV is Where the Camera Lighting is in the Tower

In this embodiment the lighting source is in the tower and not in the protrusion outline in FIG. 1 (2A). This configuration has an advantage that it is entirely contained in the tower and so easier to protect from infection risk aspect.

The lighting from 2A is alignment with the bridge of the speculum. The lighting from 2A can be a single LED or a multitude of LEDs. The lighting system is in direct line of the bridge which conducts light to the speculum. There may be a layer between the lighting system and the bridge that helps to focus or intensify the light down the bridge.

The LEDS can be powered by electronics from the tower and can be controlled by a for dimming/brightening purposes.

### FIG. 3: An Improvement of the Camera Which is on a Flex-Wire

A classical camera sensor is attached and wired to an electronics board. However, there are electronics boards where the camera sensor can be off mainboard and be positioned at the apertures.

This embodiment of the camera means that it can avoid any reflective mirrors. The aperture of the camera can be smaller as a result which results in a smaller tower.

One of the advantages of a flex-camera system is that it's a mirror-less/minimum mirrors but not necessarily lens-less system. Having a mirror system means there must be alignment of the mirrors. In the Device-GWMV system one embodiment has a mirror system to get image to the camera. However, having housing for the mirror system creates some physical restrictions on the device. One way of holding the Device-GWMV is to hold it like a pencil, where in the non-dominant hand the edge of the speculum is held with fingers and thumb for control. However the housing for the mirror system can get in the way for some hands -making it uncomfortable to use.

Having a mirror-less/minimum mirror system allows mechanism to be developed, allows a housing that can adapt the size of the user's hands. We describe a hinge like mechanism along the housing to accommodate the size of the users hands. (FIG. 3B). In the shown embodiment (but not restricted to this) the hinge can be at the base of the tower and the electronics to allow a swinging mechanism out to accommodate the size of the hand. The hinge can be strengthened since its envisage it will need to tolerate thousands of swings. The hand size can determine how far the swing mechanism is for hinge to accommodate the hand.

We would also like to describe a dynamic/intelligent illumination/video system as similarly described in FIG. 1.

In this dynamic learning system features include:
Variable illumination including the reduction and boosting of the lighting LEDs according the light feedback to the camera sensor. This can be particularly useful for views near the speculum which are at risk of being “over-illuminated” and poor image interpretation by the software. Typically to prevent overflooding of the camera sensor, digital cameras have a AGC (Automatic Gain Control) however this can take time and may not function.
Though the system is designed to be automatically—we also propose a mechanism such as a switch to manually vary the light intensity.
Integration of infrared sensors that integrate with the light sensors to optimise the clarity of imaging
A stereoscopic element can sit over the camera sensor and lighting as an alternative to create 3D vision.
Live/near-simultaneous image modification/manipulation by the interpreting software to optimise viewing.
The ability to zoom in/zoom out of a live image and the physical controls that allows to do that on the Device-GWMV or display unit.
In addition, to the software manipulation—an additional feature can be for automatic recognition of the images which may be relayed back to software which can also control/manage the patient record, remove image viewing by professionals.

Other features of the improved new device include:
Integration with the anti-infective measures as listed in the April 2017 application to prevent contamination of the LEDS during the microsuction procedure
Multicoloured LEDS where the light can be blended to form a colour of light that optimal to viewing human tissues
A control mechanism to control the brightness of the light can be controlled by the non-dominant hand or the tablet that may dim or brighten the light, turn some LEDS off or a mixture of both.
A filter mechanism in front the LEDS to filter unwanted wave lengths of light that are impede good image quality
A silencer device—if using in particular an autofocus camera, the camera will be automatically adjusting itself. In closed space of the ear, that can create unwanted noise that maybe irritating to the patient.
A sound emitting device that can be used as a form of pure tone audiometry and may even use the sound of the camera as part of that testing
A ribbed edge to the handheld portion of the speculum that allows for better handling of the speculum.
A gyroscope and associated software to correct for issues with orientation when rotating the camera that is ideally part of the internal electronics. It is important for practitioners that they are fully aware of the special geometry of the tympanic membrane. One of the original issues of the GWMV-Device is that if you turn the camera, the orientation of image seen on the camera will change as well.
A fan and cooling system that allows the electronics to stay cool during its use. This may be connected the outer housing the device to allow the outer housing to cool and feel cool during use. The outer housing is also of such a shape to be able to be received by a cover which would help to insulate the users hand away from heat.
In FIG. 10 of GWMV's UK Application GB2562901—we describe a smartphone embodiment of the GWMV-Device which has features such as a gap between the smartphone and the speculum attachment to allow instruments to pass in this space and to go through the speculum to allow procedures such as microsuction to be done.
   One of the issues of this embodiment it can be very bulky and may need a handle to securely use the device. However, the typical procedure of microsuction needs manipulation by pulling of the ear lobe to straighten the canal.
   We propose a clip like device that attaches to the devices and simultaneous attachment to the ear lobe such as soft clamp and gentle tension can be created by device to pull the ear canal to straighten it. Tension can be created but limited to by ratchet mechanism or graded length mechanism (similar to changing the length of walking sticks)

We propose various embodiments of the improved device that may have all or part of the features proposed above.

1. A speculum for use in an otoscope, wherein:
the speculum comprises a body having a passageway therethrough, defining a first end with a first opening, and a second end with a second opening;
the speculum further comprising a light source;
the body of the speculum comprising a light transmitting portion;
in which the light source is coupled to the light transmitting portion at at least one coupling point such that the light transmitting portion is arranged to transmit light through the body from each coupling point to the second end of the speculum.

2. The speculum of claim 1, in which each coupling point is at the first end of the speculum.

3. The speculum of claim 1, in which the light transmitting portion comprises a transparent material.

4. The speculum of claim 3, in which the transparent material is covered with a cladding material.

5. The speculum of claim 1, in which the light transmitting portion has a first cross-sectional area at least one coupling point, and a second cross-sectional area at the second end, with the second cross-sectional area being smaller than the first cross-sectional area.

6. The speculum of claim 1, comprising at least one light sensor arranged to determine where light is being emitted from the second end, and a control circuit coupled to each light sensor, the control circuit being arranged to modulate the brightness of the light emitted by the light source in response to light levels detected by each light sensor.

7. The speculum of claim 6, comprising a digital camera arranged to capture images through the passageway through the body of the speculum.

8. The speculum of claim 7, in which the digital camera forms the light sensor.

9. The speculum of claim 6, in which the light source comprises a plurality of light generating elements and the control circuit is arranged so as to dim or extinguish any light generating element to a region where each light sensor indicates no light is being emitted.

10. The speculum of claim 7, in which the digital camera comprises an image sensor and image processing circuitry and the speculum comprises a housing formed of first and second parts pivotably mounted with respect to each other, with first part housing the image sensor and the second part housing the image processing circuitry.

11. The speculum of claim 10, in which the first part is provided with a viewing window to view the passageway through the body of the speculum.

12-17. (canceled)

18. The speculum of claim 7, comprising an orientation sensor and a camera output circuit arranged to output an image captured from the digital camera, in which the camera output circuit has an input from the orientation sensor and is arranged to the image output from the camera output circuit based upon the input from the orientation sensor so as to correct for issues with orientation.

19. The speculum of claim 18, in which the orientation sensor comprises a gyroscope.

20. The speculum of claim 18 in which the camera output circuit is arranged so as to keep the image output consistently oriented as the speculum is rotated.

21. The speculum of claim 18 in which the camera output circuit is arranged so as to flip the image through an angle once the device has been rotated through the angle from a datum position.

22. The speculum of claim 21, in which the angle is 90 or 180 degrees.

23. The speculum of claim 7, in which the camera is provided with a focusing mechanism which allows a focus of the camera to be varied.

24. The speculum of claim 23, in which the focusing mechanism is arranged to be manually operated by a user.

25. The speculum of any preceding claim, including a cooling system, such as a fan.

26. An otoscope, comprising a digital camera, a speculum in accordance with claim 1 and an attachment means for attaching the camera to the speculum, and wherein the attachment means is for attaching to the first end of the speculum.

27-29. (canceled)
